(19) United States
(12) Patent Application Publication
(10) Pub. No.: US 2016/0289594 A1
(43) Pub. Date: Oct. 6, 2016

(54) USE OF CNGA2 AGONISTS FOR ENHANCING THE OLFACTORY EFFECT OF AN ODORANT

(71) Applicant: Henkel AG & Co. KGaA, Duesseldorf (DE)

(72) Inventors: Ursula Huchel, Koeln (DE); Manuela Materne, Kaarst (DE); Isabelle Levert, Duisburg (DE); Hubert Smyrek, Krefeld (DE); Ralf Bunn, Duesseldorf (DE); Frank Rittler, Duesseldorf (DE); Dietmar Krautwurst, Mainburg (DE); Christiane Geithe, Freising (DE)

(73) Assignee: Henkel AG & Co. KGaA, Duesseldorf (DE)

(21) Appl. No.: 15/183,356

(22) Filed: Jun. 15, 2016

Related U.S. Application Data
(63) Continuation of application No. PCT/EP2014/076883, filed on Dec. 8, 2014.

(30) Foreign Application Priority Data
Dec. 19, 2013 (DE) 10 2013 226 602.2

(51) Int. Cl. C11B 9/00; A61L 9/01; A61Q 19/10; A61K 8/34; C11D 3/50; A61K 8/49

(52) U.S. Cl. CPC C11B 9/0015; C11D 3/50; A61K 8/492; A61Q 19/10; A61K 8/342; A61K 8/347; A61K 8/34; C11B 9/0061; A61L 9/01

(57) ABSTRACT

The present invention relates to the use of compounds which are effective as agonists of the human olfactory ion channel CNGA2 (cyclic nucleotide-gated channel alpha 2) for enhancing the olfactory effect of other odorants, as well as to compositions and agents containing these compounds in combination with other odorants and to the use thereof.

### FIELD OF THE INVENTION

The present invention generally relates to odorants, as they are used in detergents or cleaning agents, cosmetic agents and air care agent, for example. The invention relates to the use of compounds that are effective as agonists of the human olfactory ion channel cyclic nucleotide-gated channel alpha 2 (CNGA2; NBI Reference Sequence: NP_005131.1) for enhancing the olfactory effect of other odorants, to compositions and agents that contain these compounds in combination with other odorants, and to the use thereof.

### BACKGROUND OF THE INVENTION

Detergents or cleaning agents, cosmetic agents such as deodorants, or air care agents (such as air freshener, air deodorizer, air spray and the like) usually include aromatic substances that impart a pleasant odor to the agents and at the same time are intended to mask malodors from an olfactory perspective. The aromatic substances usually also mask the odor of other ingredients, thereby creating a pleasant odor impression in the consumer.

The use and amount of dosage of such odorants is in particular limited by the price. Products in certain categories moreover are concentrated, as a result of which the amount of odorant or odorant composition per use is reduced. The reduction can also change the performance of the odorant, or of the odorant composition, during and after use.

To counteract a negative change and lower costs for odorants, there is a need for compositions that exhibit a comparable or stronger performance at a lower dosage.

The inventors have now surprisingly found that this object can be achieved by using a compound that is effective as an agonist of the human olfactory ion channel cyclic nucleotide-gated channel alpha 2 (CNGA2; NCBI Reference Sequence: NP_005131.1; Accession NP_005131 XP_372263; Version NP_005131.1 G1:4271.8011; Apr. 18, 2013). This is because it was found that such compounds are able to enhance the overall olfactory impression of an odorant or of an odorant mixture. It is assumed, without being limited in any way to this assumption, that CNGA2 agonists are able to directly activate ion channels of the olfactory receptor cells, and thereby lower the odor threshold values of other odorants. This hypothesis is based on experiments conducted with skatole (3-methylindole), which showed that skatole, in addition to specific odor receptors, also directly activates the CNGA2 ion channel of all odor receptor cells.

The CNGA2 ion channel is a cAMP-activated calcium channel, which plays an important role in the transduction of signals by way of odor receptors. When odors are perceived, G-protein-coupled transmembrane receptors (GPRC) are activated by the aromatic substances, which, in turn, intracellularly activate the adenylate cyclase (AC) enzyme, which then produces the messenger cAMP. cAMP, in turn, activates further intracellular signal molecules, such as ion channels and certain kinases, which ultimately results in an intracellular signal.

Enhancing the activation of the CNGA2 ion channel using skatole and related indole compounds effective as agonists of the CNGA2 channel is accordingly a suitable approach for lowering the odor threshold values of other odors. The use of such agonists of the CNGA2 channel thus results in an enhancement of the olfactory effect of odorants co-formulated therewith. The use of such compounds thus achieves the object of creating strong scents, in olfactory terms, while also considerably reducing the concentration of use of the actual odorant.

Furthermore, other desirable features and characteristics of the present invention will become apparent from the subsequent detailed description of the invention and the appended claims, taken in conjunction with this background of the invention.

### BRIEF SUMMARY OF THE INVENTION

Use of a compound that is effective as an agonist of the human olfactory ion channel cyclic nucleotide-gated channel alpha 2 (CNGA2; NCBI Reference Sequence: NP 005131.1) for enhancing the olfactory effect of an odorant.

An odorant composition, characterized by comprising at least one compound that is effective as a CNGA2 agonist, in particular in an amount between 0.000001 and 10 wt. %, advantageously between 0.0005 and 5 wt. %, more advantageously between 0.001 and 2 wt. %, and in particular between 0.001 and 1 wt. %, each based on the total composition; and at least one odorant, in particular in an amount between 1 and 99.9999 wt. %, advantageously between 10 and 99.9999 wt. %, more advantageously between 20 and 99.9999 wt. %, and in particular between 50 and 99.9999 wt. %, each based on the total composition.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In a first aspect, the invention is directed to the use of a compound that is effective as an agonist of the human olfactory ion channel CNGA2 for enhancing the olfactory effect of an odorant.

Another aspect relates to an odorant composition comprising

(a) at least one compound that is effective as a CNGA2 agonist, in particular in an amount between 0.000001 and 10 wt. %, advantageously between 0.0005 and 5 wt. %, more advantageously between 0.001 and 2 wt. %, and in particular between 0.001 and 1 wt. %, each based on the total composition; and

(b) at least one odorant, in particular in an amount between 1 and 99.9999 wt. %, advantageously between 10 and 99.9999 wt. %, more advantageously between 20 and 99.9999 wt. %, and in particular between 50 and 99.9999 wt. %, each based on the total composition.

A further aspect is directed to an agent that includes the odor composition described herein, wherein the agent is a detergent, a cleaning agent, an air care agent or a cosmetic agent.

All quantities indicated in connection with the agents described herein refer to wt. %, in each case based on the total weight of the agent, unless indicated otherwise. Moreover, quantities that relate to at least one component always relate to the total amount of this type of component present in the agent, unless explicitly indicated otherwise. This means that such quantities, for example in connection with “at least one odorant,” refer to the total amount of odorants present in the agent.

“At least one,” as used herein, refers to 1 or more, for example 1, 2, 3, 4, 5, 8, 9, or more. In connection with components of the compositions described herein, this information does not refer to the absolute amount of molecules, but to the type of the component. “At least one odorant” therefore signifies, for example, one or more different odorants, which is to say one or more different types of odorants. Together with quantities, the quantities refer to the total amount of the correspondingly identified type of component, as already defined above.

In various embodiments, the compound that is effective as a CNGA2 agonist is a compound of formula (I)

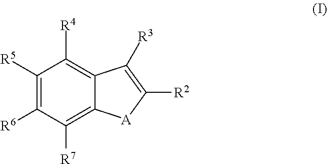

in which
A denotes a group NR¹ or an oxygen atom; and
R¹, R², R³, R⁴, R⁵, R⁶ and R⁷, independently of one another, each denote hydrogen or a linear or branched, substituted or unsubstituted alkyl group having 1 to 4 carbon atoms.

Depending on the selection of the group A, these are thus compounds of indole or compounds of benzofuran.

It is preferred according to the invention when A according to formula (I) denotes the group NR¹.

It is preferred that only one of the groups R1, R2, R3, R4, R5, R6 and R7 denotes a linear or branched, substituted or unsubstituted alkyl group having 1 to 4 carbon atoms, and the remaining groups denote hydrogen.

In preferred embodiments, the compound that is effective as a CNGA2 agonist is selected from one or more compounds from the group consisting of indole, 2-methylindole, 3-methylindole, 4-methylindole, 5-methylindole, 6-methylindole, 7-methylindole, benzofuran, 2-methylbenzofuran, 3-methylbenzofuran, 4-methylbenzofuran, 5-methylbenzofuran, 6-methylbenzofuran, and 7-methylbenzofuran, in particular 3-methylindole, 4-methylindole, 3-methylbenzofuran and 2-methylbenzofuran. The respective described indole derivatives are preferred.

The term “agonist”, as used herein in connection with the CNGA2 ion channel, denotes compounds that bind to the CNGA2 ion channel and cause the same to be activated. The affinity of the agonists is preferably sufficiently high to allow them to bind effectively to the CNGA2 channel and activate the same. In various embodiments, suitable agonists thus have a K_{d} value of at least 10 μM, preferably at least 1 μM, still more preferably at least 0.1 μM, and most preferably at least 0.01 μM. The K_{d} value describes the concentration at which 50% of the CNGA2 ion channels are present in the complex with the corresponding agonist. The K_{d} value can be determined using methods known in the prior art, for example by way of isothermal titration calorimetry (ITC), surface plasmon resonance (SPR) or fluorescence spectroscopy (measurements are each carried out at 20° C.). For example, the following determination method using the HeLa/Olf cell line (Shirokova et al., 2005, J. Biol. Chem, 280 (12): 11807-15; DE 10350054 A1) is suited, which expresses the ortholog ion channel CNGA2 from bovine (Bos taurus) in a stable manner. CNGA2 is not only the receptor for the tested compounds, but also the effector/signal generator, which, serving as the ion channel after activation, transmits an extracellular Ca²⁺ signal into the cells, which can be detected intracellularly by way of fluorescence spectroscopy using the calcium-sensitive fluorescent dye Fluo-4. The cells are brought in contact with rising concentrations of the tested compound in the presence of the fluorescent dye, and the resulting fluorescence signal is read out.

The CNGA2 agonists described herein are preferably formulated together with aromatic substances. The aromatic substances are not subject to any restrictions whatsoever and comprise aromatic substances of natural or synthetic origin, for example. These aromatic substances can be more volatile aromatic substances, aromatic substances having higher boiling points, solid aromatic substances and/or tenacious aromatic substances. It is likewise possible to use all aromatic substances disclosed herein in the form of precursor compounds. Corresponding compounds are known in the prior art.

The terms “odorant” and “aromatic substance” are used interchangeably herein and refer in particular to substances that have a scent that is perceived to be pleasant by humans. The CNGA2 agonists described herein are typically not odorants or aromatic substances within the meaning of the invention since they do not have a scent that is perceived as pleasant by humans.

It is possible, for example, to use individual odorant compounds, such as synthetic products of the ester, ether, aldehyde, ketone, alcohol, and hydrocarbon types, as aromatic substances. Odorant compounds of the ester type are, for example, benzyl acetate, phenoxyethyl isobutyrate, p-tert.-butylcyclohexyl acetate, linalyl acetate, dimethyl benzyl carbinyl acetate (DMBCA), phenylethyl acetate, benzyl acetate, ethyl methylphenyl glycinate, allyl cyclohexyl propionate, styrallyl propionate, benzyl salicylate, cyclohexyl salicylate, floramate, melusate, and jasmecyclate. The ethers include, for example, benzyl ethyl ethers and ambroxide, the aldehydes include, for example, linear alkanals having 8 to 18 carbon atoms, citral, citronellal, citronellyl oxy acetaldehyde, cyclamen aldehyde, lilial and bourgeonal, the ketones include, for example, damascones, ionones, α-isomethyl ionone and methyl cedryl ketone, the alcohols include anethol, citronellol, eugenol, geraniol, phenylethyl alcohol and terpineol, and the hydrocarbons primarily include the terpenes such as limonene and pinene. Preferably, however, mixtures of different odorants are used, which together produce an appealing odorous note.

Such mixtures can contain natural odorous substance mixtures such as those accessible from plant sources, for example pine, citrus, jasmine, patchouli, rose, or ylang ylang oil. Likewise suitable are muscatel sage oil, chamomile oil, clove oil, lemon balm oil, mint oil, cinnamon leaf oil, lime blossom oil, juniper berry oil, vetiver oil, olibanum oil, galbanum oil, and labdanum oil, as well as orange blossom oil, neroli oil, orange peel oil, and sandalwood oil.

The general description of aromatic substances that can be used (see above) generally represents the different substance classes of odorants. If it is to be perceptible, an odorant must be volatile, wherein, in addition to the nature of the functional groups and the structure of the chemical compound, the molar mass also plays an important role. Most odorants have molar masses of up to approximately 200 daltons, while molar masses of 300 daltons and above tend to be the exception. Due to the differing volatility of odorants, the odor of a perfume or aromatic substance composed of multiple odorants varies over the course of vaporization, wherein the odor impressions are divided into “top note”, “middle note or body” and “end note or dry out.” Since the perception of odor, to a large degree, is also based on the intensity of the odor, the top note of a perfume or odorant is not solely composed of highly volatile compounds, while the end note is predominantly composed of less volatile, which is to say tenacious, odorants. For example, more volatile odorants can be bound to certain fixatives in the composition of perfumes, whereby excessively fast vaporization of the same is prevented. The division of the odorants into “more volatile” and “tenacious” odorants below thus provides no information about the impression of the odor and whether the corresponding odorant is perceived as a top note or middle note.

Tenacious odorants that can be used within the scope of the present invention are, for example, essential oils such as angelica root oil, anise oil, arnica blossom oil, basil oil, bay oil, champaca flower oil, silver fir oil, silver fir cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemon grass oil, lime oil, mandarin oil, lemon balm oil, ambrette seed oil, myrrh oil, clove oil, neroli oil, niaouli oil, olibanum oil, origanum oil, palmarosa oil, patchouli oil, Peru balsam oil, petitgrain oil, pepper oil, peppermint oil, pimento oil, pine oil, rose oil, rosemary oil, sandalwood oil, celery oil, spike oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil, and cypress oil. Higher-boiling or solid odorants of natural or synthetic origin may, however, also be used within the scope of the present invention as tenacious odorants or odorant mixtures. These compounds include the compounds described below and mixtures thereof acedyl, acetophenone, ambrettolide, ambroxide, α-amylcinnamaldehyde, anethole, anisaldehyde, anise alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formiate, benzyl valerianate, borneol, bornyl acetate, boisambrene forte, bourgeonal, α-bromostyrene, n-cedryl acetate, citrusal, cyclamen aldehyde, decalactone, n-decyl aldehyde, dihydromyrcenol, dimetol, dimethyl anthranilate, dipentene, n-dodecyl aldehyde, ethyl phenylacetate, eugenol, eugenol methyl ether, eucalyptol, farnesol, fenchone, fenchyl acetate, floralozone, galaxolide, geranyl acetate, geranyl formiate, geranial, geranonitrile, helional, heliotropin, methyl heptine carbonate, heptaldehyde, hydroquinone dimethyl ether, hydroxycitronellal, hydroxycinnamaldehyde, hydroxycinnamyl alcohol, indole, irone, isoeugenol, isoeugenol methyl ether, isononyl alcohol, isosafrole, lyra, jasmone, camphor, carvacrol, carvone, kephalis, p-cresol methyl ether, coumarin, p-methoxyacetophenone, menthol, methyl N-amyl ketone, menthone, methylanthranilic acid methyl ester, p-methylacetophenone, methyl chavicol, p-methyl quinoline, methyl-β-naphthyl ketone, methyl-n-nonyl acetaldehyde, methyl-n-nonyl ketone, muscone, β-naphthol ethyl ether, β-naphthol methyl ether, neranial, nerol, n-nonyl aldehyde, nonyl alcohol, n-octyl aldehyde, p-oxyacetophenone, pentadecanolide, β-phenyl ethyl alcohol, phenylacetaldehyde dimethyl acetal, phenylacetic acid, propydyl, pulegone, safrole, isoamyl salicylate, methyl salicylate, hexyl salicylate, cyclohexyl salicylate, santalol, sandelice, skatole, tetrahydrolinanool, terpineol, thymene, thymol, triplal, troenan, γ-undecalactone, vanillin, veratrum aldehyde, cinnamaldehyde, cinnamyl alcohol, cinnamic acid, ethyl cinnamate, benzyl cinnamat.

More volatile odorants include in particular lower-boiling odorants of natural or synthetic origin, which may be used alone or in mixtures Examples of more volatile odorants are diphenyloxide, limonene, linalool, linalyl acetate and propionate, melusate, menthol, menthone, methyl-n-heptenone, pinene, phenylacetaldehyde, terpinyl acetate, citral, citronellal.

The compound that is effective as a CNGA2 agonist can be used in the odorant compositions described herein in amounts between 0.0001 and 10 wt. %, advantageously between 0.001 and 5 wt. %, more advantageously between 0.01 and 2 wt. %, and in particular between 0.1 and 1 wt. %, each based on the total composition. The amount is preferably selected such that the concentration of the CNGA2 agonist, the odor of which is typically perceived to be unpleasant by humans, is below the odor threshold, which is to say the concentration is below, and preferably considerably below, the amount that can be perceived in olfactory terms by the predominant majority of humans. At the same time, however, the concentration is sufficiently high to ensure the effect as an enhancer of the odorous experience of other aromatic substances. Concentrations that can be used by way of example in odorant compositions range from 0.001 to 0.05 wt. % CNGA2 agonist, in particular 3-methylindole, based on the odorant composition.

The amount of odorant in such odorant compositions is preferably between 1 and 99.9999 wt. %, advantageously between 10 and 99.9999 wt. %, more advantageously between 20 and 99.9999 wt. %, and in particular between 50 and 99.9999 wt. %, based on the total composition. The at least one aromatic substance is preferably a mixture of different aromatic substances, usually a mixture of 2 or more, 3 or more, 5 or more, or 10 or more aromatic substances. The concentration is selected such that the composition has the desired scent for the intended use and ensures a pleasant odorous experience.

The odorant compositions described herein and the CNGA2 agonists per se, in particular in combination with odorants, can be present in different agents, to which the invention also relates. Such agents include, but are not limited to, detergents, cleaning agents, air care agents, and cleaning agents.

The odorant composition can preferably be present in such agents in the amounts that are customary for aromatic substances/perfumes. In various embodiments, depending on the type of the agent, these are amounts between 0.0001 and 5 wt. %, advantageously between 0.001 and 4 wt. %, more advantageously between 0.01 and 3 wt. %, and in particular between 0.1 and 2 wt. %, each based on the total agent.

In addition to the described aromatic substances, the detergents and cleaning agents can, of course, include customary ingredients of such agents. In this regard, primarily surfactants, builder substances, bleaching agents, enzymes, and other active substances should be mentioned. The essential ingredients of detergents and cleaning agents include in particular surfactants.

Depending on the intended purpose of the agents according to the invention, the surfactant content will be selected higher or lower. Usually, the surfactant content of detergents ranges between 10 and 40 wt. %, preferably between 12.5 and 30 wt. %, and in particular between 15 and 25 wt. %, while cleaning agents for automatic dishwashing contain between 0.1 and 10 wt. %, preferably between 0.5 and 7.5 wt. %, and in particular between 1 and 5 wt. % surfactants.

These surface-active substances come from the group of anionic, non-ionic, zwitterionic or cationic surfactants, wherein anionic and non-ionic surfactants are clearly preferred for economical reasons and due to the performance spectrum thereof during washing and cleaning.

Anionic surfactants that are used are those of the sulfonate and sulfate types, for example. Surfactants of the sulfonate type that can be used are preferably C₉₋₁₃ alkylbenzene sulfonates, olefin sulfonates, which is to say mixtures of alkene and hydroxyalkane sulfonates, and disulfonates, as they are obtained, for example, from C₁₂₋₁₈ monoolefins having a terminal or internal double bond by way of sulfonation with gaseous sulfur trioxide and subsequent alkaline or acid hydrolysis of the sulfonation products. Also suited as alkane sulfonates obtained from C₁₂₋₁₈ alkanes, for example by way of sulfochlorination or sulfoxidation with subsequent hydrolysis or neutralization. Likewise, the esters of α-sulfofatty acids (ester sulfonates) are suitable, for example the α-sulfonated methyl esters of hydrogenated coconut, palm kernel or tallow fatty acids.

Sulfated fatty acid glycerol esters are further suitable anionic surfactants. Fatty acid glycerol esters shall be understood to mean the monoesters, diesters and triesters and the mixtures thereof, as they are obtained during production by way of the esterification of a monoglycerol with 1 to 3 moles fatty acid or during the transesterification of triglycerides with 0.3 to 2 moles glycerol. Preferred sulfated fatty acid glycerol esters are the sulfation products of saturated fatty acids having 6 to 22 carbon atoms, for example of caproic acid, caprylic acid, capric acid, myristic acid, lauric acid, palmitic acid, stearic acid or behenic acid.

The alkali salts, and in particular the sodium salts of the sulfuric acid half-esters of C₁₂ to C₁₈ fatty alcohols, for example from coconut fatty alcohol, tallow fatty alcohol, lauryl, myristyl, cetyl or stearyl alcohol, or of C₁₀ to C₂₀ oxoalcohols and the half-esters of secondary alcohols having this chain length are preferred as alk(en)yl sulfates. Furthermore preferred are alk(en)yl sulfates having the described chain length that include a synthetic straight-chain alkyl group produced on a petrochemical basis, and that have a similar degradation behavior as the adequate compounds based on fatty chemical raw materials. From a washing perspective, the C₁₂ to C₁₆ alkyl sulfates, C₁₂ to C₁₅ alkyl sulfates, and C₁₄ to C₁₅ alkyl sulfates are preferred.

The sulfuric acid monoesters of straight-chain or branched C₇₋₂₁ alcohols ethoxylated with 1 to 6 moles of ethylene oxide, such as 2-methyl-branched C₉₋₁₁ alcohols including, on average, 3.5 moles ethylene oxide (EO) or C₁₂₋₁₈ fatty alcohols including 1 to 4 EO, are also suited. Due to the high foaming behavior, they are used only in relatively small amounts in cleaning agents, for example in amounts of 1 to 5 wt. %.

Further suitable anionic surfactants are also the salts of alkyl sulfosuccinic acid, Which are also referred to as sulfosuccinates or as sulfosuccinic acid esters and represent monoesters and/or diesters of sulfosuccinic acid with alcohols, preferably fatty alcohols, and in particular ethoxylated fatty alcohols. Preferred sulfosuccinates contain C₈₋₁₈ fatty alcohol groups or mixtures of these. In particular, preferred sulfosuccinates contain a fatty alcohol group that is derived from ethoxylated fatty alcohols, which taken alone represent non-ionic surfactants (for description see below). Among these, in turn, sulfosuccinates including fatty alcohol groups that derive from ethoxylated fatty alcohols exhibiting a restricted distribution of homologs are particularly preferred. Likewise, it is also possible to use alk(en)yl succinic acid having preferably 8 to 18 carbon atoms in the alk(en)yl chain, or the salts thereof.

Further anionic surfactants that can also be used are in particular soaps. Saturated fatty acid soaps are suitable, such as the salts of lauric acid, myristic acid, palmitic acid, stearic acid, hydrogenated erucic acid and behenic acid, and in particular soap mixtures derived from natural fatty acids, such as coconut, palm kernel or tallow fatty acids.

The anionic surfactants, including the soaps, can be present in the form of the sodium, potassium or ammonium salts thereof, or as soluble salts of organic bases, such as monoethanolamine, diethanolamine or triethanolamine. The anionic surfactants are preferably present in the form of the sodium, potassium or magnesium salts thereof, and in particular in the form of the sodium salts.

There are no general conditions that must be adhered to that would stand in the way of selecting the anionic surfactants. Preferred agents, however, have a soap content that exceeds 0.2 wt. %, based on the total weight of the detergent and cleaning agent produced in step d). The use of alkylbenzene sulfonates and fatty alcohol sulfates as anionic surfactants is preferred, wherein preferred shaped detergent bodies contain 2 to 20 wt. %, preferably 2.5 to 15 wt. %, and in particular 5 to 10 wt. % fatty alcohol sulfate(s), in each case based on the weight of the agents.

Alkoxylated, advantageously ethoxylated, in particular primary alcohols having preferably 8 to 18 carbon atoms and on average 1 to 12 moles ethylene oxide (EO) per mole of alcohol, in which the alcohol residue can be linear or preferably methyl-branched at the 2-position, or can contain linear and methyl-branched residues in the mixture, such as those usually present in oxo alcohol groups, are preferably used as non-ionic surfactants. However, alcohol ethoxylates having linear groups of alcohols of native origin having 12 to 18 carbon atoms, for example of coconut, palm, tallow fatty or oleyl alcohol, and an average of 2 to 8 EO per mole of alcohol are particularly preferred. The preferred ethoxylated alcohols include, for example, C₁₂₋₁₄ alcohols having 3 EO or 4 EO, C₉₋₁₁ alcohol having 7 EO, C₁₃₋₁₅ alcohols having 3 EO, 5 EO, 7 EO, or 8 EO, C₉₋₁₁ is alcohols having 3 EO, 5 EO, or 7 EO, and mixtures thereof, such as mixtures of C₁₂₋₁₄ alcohol having 3 EO and C₁₂₋₁₈ alcohol having 5 EO. The degrees of ethoxylation indicated represent statistical averages that can correspond to an integer or a fractional number for a specific product. Preferred alcohol ethoxylates exhibit a restricted distribution of homologs (narrow range ethoxylates, NRE). In addition to these non-ionic surfactants, fatty alcohols having more than 12 EO can also be used. Examples of these are tallow fatty alcohol having 14 EO, 25 EO, 30 EO, or 40 EO.

Another class of preferably used non-ionic surfactants, which are used either as the only non-ionic surfactant or in combination with other non-ionic surfactants, are alkoxylated, preferably ethoxylated or ethoxylated and propoxylated fatty acid alkyl esters, preferably having 1 to 4 carbon atoms in the alkyl chain, in particular fatty acid methyl esters, as they are described in the Japanese patent application JP 58/217598, for example, or produced preferably according to the method described in the international patent application WO 90/13533 A.

Another class of non-ionic surfactants that can advantageously be used is the alkyl polyglycosides (APG). Alkyl polyglycosides that can be used have the general formula RO(G)_{z}, where R denotes a linear or branched, in particular methyl-branched at the 2-position, saturated or unsaturated aliphatic group having 8 to 22, preferably 12 to 18 carbon atoms, and G is the symbol that denotes a glycose unit having 5 or 6 carbon atoms, preferably glucose. The degree of glycosidation ranges between 1.0 and 4.0, preferably between 1.0 and 2.0, and in particular between 1.1 and 1.4. Preferably linear alkyl polyglycosides are used, which is to say alkyl polyglycosides in which the polyglycol group is a glucose group and the alkyl group is an n-alkyl group.

Non-ionic surfactants of the amine oxide type, for example N-cocoalkyl-N—N-dimethylamine oxide and N-tallowalkyl-N,N-dihydroxyethylamine oxide, and of the fatty acid alkanolamide type may also be suitable. The quantity of these non-ionic surfactants is preferably no more than that of the ethoxylated fatty alcohols, in particular no more than half thereof.

Further suitable surfactants are polyhydroxy fatty acid amides of formula (III),

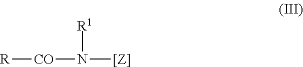

in which RCO denotes an aliphatic acyl group having 6 to 22 carbon atoms, R¹ denotes hydrogen, an alkyl or hydroxyalkyl group having 1 to 4 carbon atoms, and [Z] denotes a linear or branched polyhydroxyalkyl group having 3 to 10 carbon atoms and 3 to 10 hydroxyl groups. The polyhydroxy fatty acid amides are known substances that can usually be obtained by the reductive amination of a reducing sugar with ammonia, an alkyl amine or an alkanol amine, and subsequent acylation with a fatty acid, a fatty acid alkyl ester or a fatty acid chloride.

The group of polyhydroxy fatty acid amides also includes compounds of formula (IV),

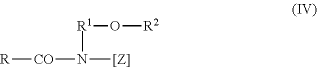

in which R denotes a linear or branched alkyl or alkenyl group having 7 to 12 carbon atoms, R¹ denotes a linear, branched or cyclic alkyl group or an aryl group having 2 to 8 carbon atoms, and R² denotes a linear, branched or cyclic alkyl group or an aryl group or an oxy alkyl group having 1 to 8 carbon atoms, wherein C₁₋₄ alkyl or phenyl groups are preferred, and [Z] denotes a linear polyhydroxy alkyl group, the alkyl chain of which is substituted with at least two hydroxyl groups, or alkoxylated, preferably ethoxylated or propoxylated derivatives of this group. [Z] is preferably obtained by the reductive amination of a reduced sugar, for example glucose, fructose, maltose, lactose, galactose, mannose or xylose. The N-alkoxy- or N-aryloxy-substituted compounds can then be converted, in the presence of an alkoxide as the catalyst, to the desired polyhydroxy fatty acid amides by reacting these with fatty acid methyl esters, for example according to the teaching of the international application WO 95/07331 A.

Builder substances are another significant group of detergents and cleaning agent ingredients. This substance class is understood to cover both organic and inorganic builder substances. These are compounds that can both perform a carrier function in the agents according to the invention and act as a water-softening substance during use.

Usable organic builder substances are, for example, the polycarboxylic acids that can be used in the form of the sodium salts thereof wherein polycarboxylic acids shall be understood to mean those carboxylic acids that carry more than one acid function. These include, for example, citric acid, adipic acid, succinic acid, glutaric acid, malic acid, tartaric acid, maleic acid, fumaric acid, saccharic acids, aminocarboxylic acids, nitrilotriacetic acid (NTA), provided that such use is not objectionable for ecological reasons, and mixtures thereof preferred salts are the salts of polycarboxylic acids such as citric acid, adipic acid, succinic acid, glutaric acid, tartaric acid, saccharic acids, and mixtures thereof in addition, the acids can also be used per se. In addition to the builder effect, the acids typically also have the property of being an acidifying component and are thus also used, for example in the granules according to the invention, as agents to set a lower and milder pH value of detergents or cleaning agents. In particular, citric acid, succinic acid, glutaric acid, adipic acid, gluconic acid and arbitrary mixtures of these shall be mentioned here.

Builders moreover include polymeric polycarboxylates; for example, these are the alkali metal salts of polyacrylic acid or of polymethacrylic acid, for example those having a relative molar mass from 500 to 70000 g/mol. This substance class was already described in detail above. The (co)polymeric polycarboxylates can be used either as a powder or as an aqueous solution. The content of (co)polymeric polycarboxylates in the agents is preferably 0.5 to 20 wt. %, and in particular 3 to 10 wt. %.

To improve water solubility, the polymers can also contain allyl sulfonic acids, such as allyloxybenzene sulfonic acid and methallyl sulfonic acid in EP 0727448 B, as a monomer. Biodegradable polymers composed of more than two different monomer units are also particularly preferred, for example those that, according to DE 4300772 A, contain salts of acrylic acid and of maleic acid, and vinyl alcohol or vinyl alcohol derivatives as monomers or, according to DE 4221381 C, salts of acrylic acid and of 2-alkylallylsulfonic acid and sugar derivatives as monomers. Further preferred copolymers are those that are described in the German patent applications DE 4303320 A and DE 4417734 A and preferably comprise acrolein and acrylic acid/acrylic acid salts or acrolein and vinyl acetate as monomers. Polymeric aminodicarboxylic acids, the salts thereof or the precursor substances thereof should likewise be mentioned as additional preferred builder substances. Polyaspartic acid or the salts and derivatives thereof are particularly preferred, of which it is disclosed in the German patent application DE 19540086 A that they also exhibit a bleach-stabilizing effect, in addition to co-builder properties.

Additional suitable builder substances are polyacetals, which may be obtained by reacting dialdehydes with polyolcarboxylic acids having have 5 to 7 carton atoms and at least 3 hydroxyl groups, for example as described in the European patent application EP 0280223 A. Preferred polyacetals are obtained from dialdehydes such as glyoxal, glutaraldehyde, terephthalaldehyde and mixtures thereof, and from gluconic acid and/or glucoheptonic acid.

Further suitable organic builder substances are dextrins, for example oligomers or polymers of carbohydrates, which can be obtained by the partial hydrolysis of starches. The hydrolysis can be carried out according to customary, for example acid- or enzyme catalyzed, methods. These are preferably hydrolysis products having average molar masses in the range of 400 to 500000 g/mol. A polysaccharide having a dextrose equivalent (DE) in the range of 0.5 to 40, and in particular of 2 to 30, is preferred, wherein DE is a customary measure for the reducing action of a polysaccharide compared to dextrose, which has a DE of 100. It is possible to use both maltodextrins having a DE between 3 and 20 and dried glycose syrups having a DE between 20 and 37, and so-called yellow dextrins and white dextrins having higher molar masses in the range of 2000 to 30000 g/mol. A preferred dextrin is described in the British patent application 9419091. The oxidized derivatives of such dextrins are the reaction products thereof with oxidizing agents, which are able to oxidize at least one alcohol function of the saccharide ring into a carboxylic acid function. Such oxidized dextrins and methods for the production thereof are known, for example, from the European patent applications EP 0232202 A, EP 0427349 A, EP 0472042 A and EP 0542496 A, and the international patent applications WO 92/18542, WO 93/08251 A, WO 93/16110 A, WO 94/28030 A, WO 95/07303 A, WO 95/12619 A and WO 95/20608 A. An oxidized oligosaccharide according to the German patent application DE 19600018 A is likewise suited. A product that is oxidized on C₆ of the saccharide ring can be particularly advantageous.

Oxydisuccinates and other derivatives of disuccinates, preferably ethylenediamine disuccinate, are further suitable cobuilders. Ethylenediamine-N,N′-disuccinate (EDDS), the synthesis of which is described in U.S. Pat. No. 3,158,615, for example, is preferably used in the form of the sodium or magnesium salts thereof glycerol disuccinates and glycerol trisuccinates, as they are described in the US patent specifications U.S. Pat. No. 4,524,009, U.S. Pat. No. 4,639,325, in the European patent application EP 0150930 A and in the Japanese patent application JP 93/339896, are also furthermore preferred in this context. Suitable amounts are 3 to 15 wt. % for zeolite-containing and/or silicate-containing formulations.

Further organic cobuilders that can be used are, for example, acetylated hydroxycarboxylic acids or the salts thereof, which optionally can also be present in lactone form and comprise at least 4 carbon atoms and at least one hydroxy group, as well as no more than two acid groups. Such cobuilders are described, for example, in the international patent application WO 95/20029 A.

A further class of substances having cobuilder properties is phosphonates. These include, in particular, hydroxyalkane and aminoalkane phosphonates. Among the hydroxyalkanephosphonates, 1-hydroxyethane-1,1-diphosphonate (HEDP) is of particular importance as a cobuilder. It is preferably used as a sodium salt, wherein the disodium salt reacts in a neutral fashion, and the tetrasodium salt acts in an alkaline manner (pH 9). Possible preferable aminoalkane phosphonates include ethylenediaminetetramethylene phosphonate (EDTMP), diethylentriaminepentamethylene phosphonate (DTPMP) and the higher homologues thereof. They are preferably used in the form of the neutrally reacting sodium salt, for example as the hexasodium salt of EDTMP or as the hepta- and octa-sodium salt of DTPMP. Of the class of phosphonates, HEDP is preferably used as a builder. The aminoalkane phosphonates additionally have a pronounced capability to bind heavy metals. Accordingly, it may preferred, in particular if the agents also include bleach, to use aminoalkanephosphonates, in particular DTPMP, or mixtures of the cited phosphonates.

Moreover, all compounds that are able to form complexes with alkaline earth ions can be used as cobuilders.

A preferably used inorganic builder is microcrystalline, synthetic and bound water-containing zeolite. The microcrystalline, synthetic and bound water-containing zeolite that is used is preferably zeolite A and/or P. However, zeolite X, and mixtures of A, X and/or P are also suited, for example a co-crystallisate of the zeolites A and X. The zeolite can be used in the form of a spray-dried powder or else as an undried, stabilized suspension that is still moist from production. In the event that the zeolite is used in the form of a suspension, the same may contain small amounts of additives of non-ionic surfactants as stabilizers, for example 1 to 3 wt. %, based on zeolite, of ethoxylated C₁₂ to C₁₈ fatty alcohols having 2 to 5 ethylene oxide groups, C₁₂ to C₁₄ fatty alcohols having 4 to 5 ethylene oxide groups, or ethoxylated isotridecanols. Suitable zeolites have a mean particle size of less than 10 μm (volume distribution; measuring method: Coulter counter) and preferably contain 18 to 22 wt. %, and in particular 20 to 22 wt. % hound water. In preferred embodiments, zeolites are present in the premix in amounts of 10 to 94.5 wt. %, Wherein it may be particularly preferred for zeolites to be present in amounts of 20 to 70, and in particular 30 to 60 wt. %.

Suitable partial substitutes for zeolites are phyllosilicates of natural and synthetic origin. Such phyllosilicates are known from patent applications DE 2334899 A, EP 0026529 A and DE 3526405 A, for example. The usability of the same is not limited to a specific composition or chemical formula. However, smectites are preferred, and in particular bentonites. Crystalline, layered sodium silicates of the general formula NaMSiₓO₂ₓ₊₁.yH₂O, in which M denotes sodium or hydrogen, x denotes a number from 1.9 to 4, and y denotes a number from 0 to 20, and preferred values for x are 2, 3 or 4, are also suitable for the substitution of zeolites or phosphates. Such crystalline phyllosilicates are described, for example, in the European patent application EP 0164514 A. Preferred crystalline phyllosilicates of the indicated formula are those in which M denotes sodium and x takes on the value 2 or 3, in particular, both β- and δ-sodium silicates Na₂Si₂O₅.yH₂O are preferred.

Preferred builder substances also include amorphous sodium silicates having a Na₂O:SiO₂ module of 1:2 to 1:3.3, preferably of 1:2 to 1:2.8, and in particular of 1:2 to 1:2.6, which exhibit retarded dissolution and secondary washing properties. The retarded dissolution compared to conventional amorphous sodium silicates can have been caused in a variety of ways, for example by way of surface treatment, compounding, compacting/compression or over-drying. Within the scope of the present invention, the term “amorphous” shall also be understood to mean “X-ray amorphous,” This means that the silicates do not supply any sharp X-ray reflexes in X-ray diffraction experiments, such as those that are typical of crystalline substances, but at best one or more maxima of the scattered X-rays, which have a width of several degree units of the diffraction angle. However, particularly good builder properties may very well also be achieved when the silicate particles supply washed-out or even sharp diffraction maxima in electron diffraction experiments. This is to be interpreted such that the products comprise microcrystalline regions measuring 10 to several hundred nm, wherein values up to a maximum of 50 nm, and in particular up to a maximum of 20 nm are preferred. Such so-called X-ray amorphous silicates, which likewise exhibit retarded dissolution compared to conventional water glasses, are described in the German patent application DE 4400024 A, for example. In particular, compressed/compacted amorphous silicates, compounded amorphous silicates, and overdried X-ray amorphous silicates are preferred, wherein in particular the overdried silicates preferably also occur as carriers in the granules according to the invention or are used as carriers in the method according to the invention.

It is also possible, of course, to use the generally known phosphates as builder substances, provided that such use should not be avoided for ecological reasons. In particular, the sodium salts of orthophosphates, pyrophosphates, and in particular of tripolyphosphates are suited. The content thereof generally does not exceed 25 wt. %, and preferably 20 wt. %, in each case based on the finished agent. In some cases it has been shown that in particular tripolyphosphates even in small amounts up to a maximum of 10 wt. %, based on the finished agent, in combination with other builder substances result in a synergistic improvement of the secondary washing performance.

In addition to the cited components, the detergents and cleaning agents according to the invention can additionally include one or more substances from the group of bleaching agents, bleach activators, enzymes, pH-setting, agents, fluorescent agents, dyes, foam inhibitors, silicone oils, anti-redeposition agents, optional brighteners, graying inhibitors, color transfer inhibitors, corrosion inhibitors, and silver protection agents. Suitable agents are known in the prior art.

This enumeration of detergent and washing agent ingredients is by no means exhaustive, but merely reflects the most essential typical ingredients of such agents. In particular, the agents may also include organic solvents, if liquid or gel-like preparations are involved. Preferably monohydric or polyhydric alcohols having 1 to 4 carbon atoms are used. Preferred alcohols in such agents are ethanol, 1,2-propanediol, glycerol, and mixtures of these alcohols. In preferred embodiments, such agents include 2 to 12 wt. % of such alcohols.

In general, the agents can be present in different states. In a preferred embodiment, the detergents or cleaning agents are liquid or gel-like agents, in particular liquid detergents or liquid dishwashing agents or cleaning gels, wherein these can in particular also be gel-like cleaning agents for flushing toilets. Such gel-like cleaning agents for flushing toilets are described, for example, in the German patent application DE 19715872 A.

Further typical cleaning agents that may include in the silyl enol ethers according to the invention are liquid or gel-like cleaners for hard surfaces, in particular those known as all-purpose cleaners, glass cleaners, floor or bathroom cleaners, and special embodiments of such cleaners, which also include acid or alkaline forms of all-purpose cleaners, as well as glass cleaners having what is known as anti-rain action. These liquid cleaning agents can be present either in one or in multiple phases. In a particularly preferred embodiment, the cleaners have two different phases.

Cleaner, in the broadest sense, is a designation for, usually surfactant-containing, formulations having a very wide range of use and, as a function thereof, a widely varying composition. The most important market segments are household cleaners, industrial (technical) and institutional cleaners. Based on the pH value, a distinction is made between alkaline, neutral and acid cleaners, and according to the form in which the product is offered, a distinction is made between liquid and solid cleaners (including in tablet form). Contrary to dishwashing agents, for example, which can likewise be categorized in the cleaner product group, what are known as cleaners for hard surfaces exhibit an optimal application profile, both in the concentrated state and in a diluted aqueous solution, in conjunction with mechanical energy. Cold cleaners develop the action thereof without an increased temperature. What is decisive for the cleaning action is above all the surfactants and/or alkali carriers, alternatively acids, optionally also solvents such as glycol ethers and lower alcohols. In general, the formulations moreover include builders, and depending on the type of cleaner also bleaching agents, enzymes, microbe-mitigating or disinfecting additives, perfume oils and dyes. Cleaners can also be formulated as microemulsions. The cleaning success, to a large degree, depends on the type of soiling, which also varies widely geographically, and the properties of the surfaces to be cleaned.

The cleaners can include anionic, non-anionic, amphoteric or cationic surfactants as the surfactant component, or surfactant mixtures of one, more or all these surfactant classes. The cleaners contain surfactants in amounts, based on the composition, of 0.01 to 30 wt. %, preferably 0.1 to 20 wt. %, in particular 1 to 14 wt. %, and extremely preferably 3 to 10 wt. %.

Suitable non-ionic surfactants in such all-purpose cleaners are, for example, C₈ to C₁₈ alkanol polyglycol ethers, alkyl polyglycosides and nitrogen-containing surfactants and mixtures thereof, in particular of the first two. The agents contain non-ionic surfactants in amounts, based on the composition, of 0 to 30 wt. %, preferably 0.1 to 20 wt. %, in particular 0.5 to 14 wt. %, and extremely preferably 1 to 10 wt. %.

C₈₋₁₈ alkanol polypropylene glycol/polyethylene glycol ethers represent known non-ionic surfactants. They can be described by the formula RⁱO—(CH₂CH(CH₃)O)ₚ(CH₂CH₂O)ₑ—H, in which R′ denotes a linear or branched aliphatic alkyl and/or alkenyl group having 8 to 18 carbon atoms, p denotes 0 or numbers from 1 to 3, and e denotes numbers from 1 to 10. The C₈₋₁₈ alkanol polyglycol ethers can be obtained by way of addition of propylene oxide and/or ethylene oxide to alkanols, preferably to fatty alcohols. Typical examples are polyglycol ethers in which R denotes an alkyl group having 8 to 18 carbon atoms, p denotes 0 to 2, and e denotes numbers from 2 to 7. Preferred representatives are, for example, C₁₀ to C₁₄ fatty alcohol+1PO+6EO ether (p=1, e=6), and C₁₂ to C₁₈ fatty alcohol+7EO ether (p=0, e=7) and the mixtures thereof.

It is also possible to use end-capped C₈ to C₁₈ alkanol polyglycol ethers, which is to say compounds in which the free OH group is etherified. The end-capped C₈₋₁₈ alkanol polyglycol ethers can be obtained according to relevant methods of preparative organic chemistry. Preferably, C₈₋₁₈ alkanol polyglycol ethers are reacted in the presence of bases with alkyl halides, in particular butyl or benzyl chloride. Typical examples are mixed ethers, in which R′ denotes a technical fatty alcohol group, preferably a C_{12/14} coconut alkyl group, p denotes 0, and e denotes 5 to 10, which are capped with a butyl group.

Furthermore, the alkyl polyglycosides already described above are preferred non-ionic surfactant.

Nitrogen-containing surfactants may be present as further non-ionic surfactants, such as fatty acid polyhydroxyamides, for example glucamides and ethoxylates of alkyl amines, vicinal diols and/or carboxylic acid amides that include alkyl groups having 10 to 22 carbon atoms, and preferably 12 to 18 carbon atoms. The degree of ethoxylation of these compounds is generally between 1 and 20, and preferably between 3 and 10. Ethanolamide derivatives of alkanoic acids having 8 to 22 carbon atoms, and preferably 12 to 16 carbon atoms, are preferred. Particularly suitable compounds include lauric acid, myristic acid and palmitic acid monoethanolamides.

Anionic surfactants suitable for all-purpose cleaners are C₈₋₁₈ alkyl sulfates, C₈₋₁₈ alkyl ether sulfates, which is to say the sulfating products of alcohol ethers, and/or C₈₋₁₈ alkylbenzene sulfonates, but also C₈₋₁₈ alkane sulfonates, C₈₋₁₈ α-olefin sulfonate, sulfonated C₈₋₁₈ fatty acids, in particular dodecylbenzene sulfonate, C₈₋₂₂ carboxylic acid amide ether sulfates, sulfosuccinic acid mono- and di-C₁₀₁₂ alkyl esters, C₈₋₁₈ alkyl polyglycol ether carboxylates, C₈₋₁₈ N-acyl taurides, C₈₋₁₈ N-sarcosinates, and C₈₋₁₈ alkyl isethionates, and the mixtures thereof. They are used in the form of the alkali metal and alkaline earth metal salts thereof, in particular sodium, potassium and magnesium salts, and ammonium- and mono-, di-, tri- or tetra-alkyl ammonium salts, and, in the case of the sulfonates, also in the form of the corresponding acid thereof, such as dodecylbenzene sulfonic acid. The agents contain anionic surfactants in amounts, based on the composition, of 0 to 30 wt. %, preferably 0.1 to 20 wt. %, in particular 1 to 14 wt. %, and extremely preferably 2 to 10 wt. %.

Due to the foam-controlling properties thereof, the all-purpose cleaners can also include soaps, which is to say alkali or ammonium salts of saturated or unsaturated C₆₋₂₂ fatty acids. The soaps can be used in an amount of up to 5 wt. %, and preferably of 0.1 to 2 wt. %.

Suitable amphoteric surfactants are, for example, betaines of formula (Rⁱⁱ)(Rⁱⁱ¹)(R^{iv})N⁺CH₂COO⁻, in which Rⁱⁱ denotes an alkyl group, which is optionally interrupted by heteroatoms or heteroatom groups, having 8 to 25, and preferably 10 to 21 carbon atoms, and Rⁱⁱⁱ and R^{iv} denote identical or different alkyl groups having 1 to 3 carbon atoms, in particular C₁₀₋₁₈ alkyl dimethyl carboxymethyl betaine and C₁₁₋₁₇ alkyl amido propyl dimethyl carboxymethyl betaine. The agents contain amphoteric surfactants in amounts, based on the composition, of 0 to 15 wt. %, preferably 0.01 to 10 wt. %, and in particular 0.1 to 5 wt. %.

Suitable cationic surfactants are, among other things, the quaternary ammonium compounds of formula (R^{v})(R^{vi})(R^{vii})(R^{viii})N⁺X⁻, in which R^{v} to R^{viii} four identical or different, and in particular two long-chain and two short-chain, alkyl groups, and X⁻ denotes an anion, and in particular a halide ion, for example didecyl dimethyl ammonium chloride, alkyl benzyl didecyl ammonium chloride and the mixtures thereof. The agents contain cationic surfactants in amounts, based on the composition, of 0 to 10 wt. %, preferably 0.01 to 5 wt. %, and in particular 0.1 to 3 wt. %.

In a preferred embodiment, the cleaners include anionic and non-anionic surfactants together with each other, preferably C₈₋₁₈ alkylbenzene sulfonates, C₈₋₁₈ alkyl sulfates and/or C₈₋₁₈ alkyl ether sulfates together with C₈₋₁₈ alkanol polyglycol ethers and/or alkyl polyglycosides, and in particular C₈₋₁₈ alkylbenzene sulfonates together with C₈₋₁₈ alkanol polyglycol ethers.

The cleaners according to the invention can moreover contain builders. Suitable builders are, for example, alkali metal gluconates, citrates, nitrilotriacetates, carbonates and bicarbonates, in particular sodium gluconate, citrate and nitrilotriacetate, and sodium and potassium carbonate and bicarbonate, and alkali metal and alkaline earth metal hydroxides, in particular sodium and potassium hydroxide, ammonia and amines, in particular monoethanolamine and triethanolamine, and the mixtures thereof these also include the salts of glutaric acid, succinic acid, adipic acid, tartaric acid and benzenehexacarboxylic acid, as well as phosphonates and phosphates. The agents contain builders in amounts, based on the composition, of 0 to 20 wt. %, preferably 0.01 to 12 wt. %, in particular 0.1 to 8 wt. %, and extremely preferably 0.3 to 5 wt. %, wherein, however, the amount of sodium hexametaphospate, excluding the agents used, is limited to 0 to 5 wt. %. Serving as electrolytes, the builder salts are auxiliary phase separation agents at the same time.

In addition to the cited components, the cleaners according to the invention may contain further auxiliary agents and additives as they are common in such agents. These include in particular polymers, active soil release substances, solvents (such as ethanol, isopropanol, glycol ether), solubilizers, hydrotropes (such as cumol sulfonate, octyl sulfate, butyl glucoside, butyl glycol), detergent boosters, viscosity regulators (such as synthetic polymers such as polysaccharides, polyacrylates, naturally occurring polymers and the derivatives thereof, such as xanthan gum, other polysaccharides and/or gelatin), pH regulators (such as citric acid, alkanol amines or NaOH), disinfectants, anti-static agents, preservatives, bleaching systems, enzymes, dyes, and opacifiers or skin protectants, as they are described in EP 0522506 A. The amount of such additives in the cleaning agent usually does not exceed 12 wt. %. The lower limit of use depends on the type of the additive and can be as much as 0.001 wt. % and below, for example, in the case of dyes. The amount of auxiliary agents preferably ranges between 0.01 and 7 wt. %, and in particular between 0.1 and 4 wt. %.

The pH value of the all-purpose cleaners can be varied across a wide range; however, a range from 2.5 to 12, and in particular from 5 to 10.5 is preferred. The pH value in the present invention shall be understood to mean the pH value of the agent in the form of the temporary emulsion.

Such all-purpose cleaner formulations can be modified for arbitrary purposes. Glass cleaners form a particular embodiment. What is essential with such cleaners is that stains or outlines remain. In particular, it is a problem that, after cleaning, water condenses on these surfaces and results in what is known as the fogging effect. It is likewise undesirable when what are known as rain stains remain on glass panes exposed to rain. This effect is known rain effect, or anti-rain effect. These effects can be prevented by suitable additives in glass cleaners.

In another preferred embodiment, the agents are powdery or granular agents. The agents according to the invention can have arbitrary bulk densities. The spectrum of possible bulk densities ranges from low bulk densities of less than 600 g/l, such as 300 g/l, through the range of average bulk densities from 600 to 750 g/l, to the range of high bulk densities of at least 750 g/l.

Arbitrary methods, which are known from the related art, are suitable for producing such agents.

The invention likewise relates to air care agents that contain the compositions according to the invention. The air care agent can be an air freshener, air deodorizer or air spray.

The present invention further relates to cosmetic agents for hair or skin treatment, which preferably include the odorant compositions described herein in the amounts already described above in connection with the other agents. In a preferred embodiment, the cosmetic agents are aqueous preparations that contain active surface-active substances and that are suitable in particular for treating keratin fibers, in particular human hair, or for treating skin.

The addressed hair treatment agents are in particular agents for treating human scalp hair. The most common agents of this category can be divided into shampoos, hair care agents, hair fixing agents and hair styling agents, as well as hair dyes and hair removal agents. The preferred agents according to the invention containing active surface-active substances include in particular shampoos and hair care agents. These aqueous preparations are typically present in a liquid to pasty form.

Fatty alcohol polyglycol ether sulfates (ether sulfates, alkyl ether sulfates), at times in combination with other usually anionic surfactants, are used predominantly for the most important group of ingredients, this being the active surface-active substances or substances that provide washing action. In addition to good cleaning power and insensitivity to water hardness, shampoo surfactants should be tolerated by the skin and mucous membranes. In accordance with statutory provisions, they must be easily biodegradable. In addition to alkyl ether sulfates, preferred agents can additionally include further surfactants such as alkyl sulfates, alkyl ether carboxylates, preferably having degrees of ethoxylation from 4 to 10, and surfactant protein/fatty acid condensates.

The goal of hair care agents is to preserve the natural state of newly regrown hair for as long as possible, and to restore the same if damaged. Features that characterize this natural state are a silky sheen, low porosity, an elastic, yet soft volume, and a pleasantly smooth feel. An important prerequisite for this is a clean, not overly oily scalp that is free of dandruff Today, a plurality of different products are covered by hair care agents, the most important representatives of which are referred to as pre-treatment agents, hair tonics, hair styling aids, hair conditioners and deep conditioning products.

The aqueous preparations for treating skin are in particular preparations for human skin care. This care begins with cleansing, for which primarily soaps are used. In this regard, a distinction is made between solid soap, usually in pieces, and liquid soap. Accordingly, in a preferred embodiment the cosmetic agents are present as shaped bodies that contain surface-active ingredients. In a preferred embodiment, the most important ingredients of such shaped bodies are the alkali salts of fatty acids of natural oils and fats, preferably having chains of 12 to 18 carbon atoms. Since lauric acid soaps lather particularly well, coconut and palm kernel oils rich in lauric acid are preferred raw materials for fine soap production. The Na salts of fatty acid mixtures are solid; the K salts are soft-pasty. For saponification, the diluted caustic soda or caustic potash is added to the fat raw materials at a stoichiometric ratio so that an excess of lye of no more than 0.05% is present in the finished soap. In many instances, soaps today are no longer produced directly from the fats, but from the fatty acids obtained by way of lipolysis. Customary soap additives are fatty acids, fatty alcohols, lanolin, lecithin, vegetable oils, partial glycerides and similar fat-like substances for lipid replenishment of the cleansed skin, antioxidants such as ascorbyl palmitate or tocopherol for preventing auto-oxidation of the soap (rancidity), complexing agents such as nitrilotriacetate for binding heavy metal traces that could catalyze the auto-oxidative spoilage, perfume oils for achieving the desired odorous notes, dyes for coloring the pieces of soap, and optionally special additives.

Liquid soaps are based on both K salts of natural fatty acids and on synthetic anionic surfactants. In aqueous solution, they contain fewer substances that provide washing action than solid soaps, and include the customary additives, optionally including viscosity-regulating components, and pearlescence additives. Due to the convenient and hygienic application from dispensers, they are preferably used in public lavatories and the like. Body washes for particularly sensitive skin are based on synthetic surfactants having a mild action, to which skin care substances are added and which are set to a neutral or slightly acidic pH (pH 5.5).

For cleansing primarily facial skin, a number of additional preparations are available, such as facial toners, cleansing lotions, milks, creams and pastes; some face packs are used for cleansing, but they generally refresh and nourish the facial skin. Facial toners are typically aqueous-alcoholic solutions having a low surfactant content and containing further skin care substances. Cleansing lotions, milks, creams and pastes are typically based on O/W emulsions that have a relatively low fatty component content and contain cleansing and nourishing additives. What are known as scruffing and peeling preparations contain substances that have a mild keratolytic effect to remove the upper horny layer of dead skin; in some instances these preparations also contain an added abrasively acting powder. Almond bran, which has been used as a mild cleansing care agent for quite some time, is frequently still a component of such preparations today. Agents for the cleansing treatment of blemished skin also contain antibacterial and anti-inflammatory substances, since the accumulation of sebaceous material in comedones (blackheads) represents a breeding ground for bacterial infections and tends cause inflammation. The wide range of different skin cleansing products offered varies in terms of the composition and content of different active ingredients depending on different skin type and specific treatment purposes.

Further preferred cosmetic agents according to the invention are agents for influencing body odor. This refers in particular to deodorizing agents. Such deodorants are able to mask, remove or destroy odors. Unpleasant body odors arise from the bacterial decomposition of sweat, in particular in the warm and moist axilla regions, where microorganisms encounter good living conditions. As a result, antimicrobial substances are the most important ingredients of deodorants. In particular, antimicrobial substances that have a substantially selective effectiveness with respect to bacteria responsible for body odor are preferred. Preferred active ingredients, however, have only a bacteriostatic effect and by no means completely destroy the bacterial flora. Antimicrobial agents include in general all suitable preservatives that specifically work against gram-positive bacterial. These are, for example, Irgasan DP 300 (triclosan, hydroxydiphenylether), chlorhexidine (1,1′-hexamethylenebis(5-(4′-chlorophenyl)-biguanide) and 3,4,4′-trichlorocarbanilide. In general, quaternary ammonium compounds are likewise suited. Due to the high antimicrobial effectiveness, all these substances are preferably used only in low concentrations of approximately 0.1 to 0.3 wt. %. Moreover, numerous odorants also exhibit antimicrobial properties. Accordingly, such odorants having antimicrobial properties are preferably used in deodorants. In particular, farnesol and phenoxyethanol shall be mentioned here. It is in particular preferred when the deodorants according to the invention include bacteriostatically acting odorants per se. The odorants can preferably be present again in the form of the odorant compositions described herein. A further group of essential ingredients of deodorants are enzyme inhibitors, which inhibit the enzymatic decomposition of sweat, such as triethyl citrate or zinc glycinate, for example. Essential ingredients of deodorants are furthermore also antioxidants, which are intended to prevent oxidation of sweat components.

In a further likewise preferred embodiment of the invention, the cosmetic agent is a hair setting agent that contains polymers for setting. It is particularly preferred if at least one polyurethane is present among the polymers.

In principle, all embodiments disclosed in connection with the use of the CNGA2 agonists can also be applied to the described odorant compositions, agents and methods, and vice versa. It goes without saying, for example, that all special CNGA2 agonists described herein can be used in the described odorant compositions, agents and methods for use thereof.

### EXAMPLES

### Example 1

A mixture of 6 aromatic substances was produced by combining equal volumes of corresponding parent solutions. The parent solutions contained 20 wt. % citronellol, 20 wt. % geraniol, 20 wt. % 2-phenylethanol, 5 wt. % 1(1,2,3,4,5,6,7,8-octahydro-2,3,8,8-tetramethyl-2-naphthyl)ethane-1-on, 1 wt. % nerol and 1 wt. % eugenol, each including dipropylene glycol as the solvent. 0.05 wt. % indole (M1) and 0.05 wt. % 3-methylindole (M2) were added to this base mixture M, the respective mixtures were applied to smelling strips, given to test persons for smelling, and the scent intensities were indicated on a scale from 1 to 10, wherein 1 represents the lowest intensity and 10 the highest intensity. The results are indicated in Table 1.

TABLE 1

| Aromatic substance | Scent intensity |
|---|---|
| M | 5 |
| M1 | 7 |
| M2 | 10 |

### Example 2

Different agents that are customary in the market (fabric softener, powdered detergent, hand dishwashing agent, shower gel), each containing conventional perfume oils as the aromatic substance, were tested in comparison with those that contained 3-methylindole as an additive in the perfume oil. As in Example 1, the respective products were given to test persons for smelling and the scent intensity was rated on a scale from 1 to 10. The results are shown in Table 2.

TABLE 2

| | Concentration of 3- | Concentration of | |
|---|---|---|---|
| | methylindole in | perfume oil in | Scent |
| Product | perfume oil (wt. %) | product (wt. %) | intensity |
| Fabric softener | — | 0.8 | 4 |
| Fabric softener | 0.03 | 0.8 | 5 |
| Powdered detergent | — | 0.3 | 3 |
| Powdered detergent | 0.005 | 0.3 | 5 |
| Hand dishwashing | — | 0.22 | 3 |
| Hand dishwashing | 0.001 | 0.20 | 3.5 |
| Hand dishwashing | 0.006 | 0.20 | 4 |
| Shower gel | — | 1 | 3.5 |
| Shower gel | 0.001 | 1 | 5 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An odorant composition, characterized by comprising
(a) at least one compound that is effective as a CNGA2 agonist, in particular in an amount between 0.000001 and 10 wt. %, advantageously between 0.0005 and 5 wt. %, more advantageously between 0.001 and 2 wt. %, and in particular between 0.001 and 1 wt. %, each based on the total composition; and
(b) at least one odorant, in particular in an amount between 1 and 999999 wt. %, advantageously between 10 and 99.9999 wt. %, more advantageously between 20 and 99.9999 wt. %, and in particular between 50 and 99.9999 wt. %, each based on the total composition.

2. An agent, characterized by comprising an odorant composition according to claim 1, the agent being a detergent, a cleaning agent, an air care agent or a cosmetic agent.

3. The agent according to claim 2, characterized in that the odorant composition is present in amounts between 0.0001 and 5 wt. % based on the total agent.

4. A method for enhancing the olfactory effect of an odorant wherein an odorant composition is combined with a compound that is effective as an agonist of the human olfactory ion channel cyclic nucleotide-gated channel alpha 2 (CNGA2; NCBI Reference Sequence: NP_005131.1).

5. A method according to claim 4, characterized in that the compound that is effective as a CNGA2 agonist is a compound of formula (I), 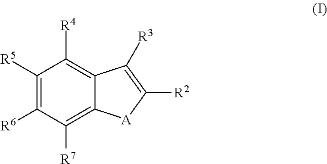
in which
A denotes a group NR¹ or an oxygen atom; and
R¹, R², R³, R⁴, R⁵, R⁶ and R⁷, independently of one another, each denote hydrogen or a linear or branched, substituted or unsubstituted alkyl group having 1 to 4 carbon atoms.

6. A method according to claim 5, characterized in that the compound that is effective as a CNGA2 agonist is selected from one or more compounds from the group consisting of indole, 2-methylindole, 3-methylindole, 4-methylindole, 5-methylindole, 6-methylindole, 7-methylindole, benzofuran, 2-methylbenzofuran, 3-methylbenzofuran, 4-methylbenzofuran, 5-methylbenzofuran, 6-methylbenzofuran, and 7-methylbenzofuran.

7. A method according to claim 4, characterized in that the compound that is effective as a CNGA2 agonist has a K_{d} value of at least 10 μM.
